(19) United States
(12) Patent Application Publication
(10) Pub. No.: US 2026/0114392 A1
(43) Pub. Date: Apr. 30, 2026

(54) APPARATUS AND METHOD FOR PROCESSING ATTRIBUTES OF SYSTEM MODULES TO DETERMINE AND/OR PREDICT AN IRRIGATION SCHEDULE

(71) Applicant: WaterPoint Irrigation Technologies, Inc., Canby, OR (US)

(72) Inventor: William A. Terry, Canby, OR (US)

(21) Appl. No.: 19/048,765

(22) Filed: Feb. 7, 2025

Related U.S. Application Data
(60) Provisional application No. 63/703,010, filed on Oct. 28, 2024.

(51) Int. Cl. A01G 25/16; G01N 33/24; G01W 1/10; G06Q 50/02

(52) U.S. Cl. CPC A01G 25/167; A01G 25/165; G01N 33/245; G01N 33/246; G01W 1/10; G06Q 50/02

(57) ABSTRACT

Disclosed are a system, method and apparatus to process attributes of electrical power signals applied for the reduction in surface tension of irrigation water. In one particular implementation, a recommended schedule for irrigation of a crop field may be determined and/or modified based, at least in part, on measurements of the attributes of electrical power signals.

This application claims the benefit of priority to U.S. Provisional Patent Appl. Ser. No. 63/703,010, titled “AI-driven irrigation decision support system,” filed Oct. 28, 2024, and incorporated herein by reference in its entirety.

### BACKGROUND

### 1. Field

This disclosure relates to methods and/or techniques for irrigation of a crop field using water treated to reduce its surface tension.

### 2. Information

In modern agriculture, farmers have moved away from flooding fields to irrigate crops using highly efficient pressurized-irrigation systems, to more precisely deliver water to the soil to feed crops. Pumping water from wells (e.g., ground water) and canals, ponds, rivers, etc. (surface water) typically requires a significant amount of energy to meet crops' irrigation demand. Labor, electrical and fuel costs to pump water from the source are quite often the largest portion of the overall costs of crop production.

With the advent of pressurized irrigation, one evolution in precision irrigation has been Center Pivot, Micro Sprinkler, and Drip Irrigation, as well as subsurface drip irrigation. Then came water-applied nutrition and chemistry to improve the accuracy and reduce the cost of applying these inputs.

Since the mid 1980's many other technologies have been introduced to, in real-time, measure and chart, via web application software and now using apps on web-enabled personal smart devices that instantly receive notifications, alerts, of out-of-boundary sensor data readings—from which farmers may more immediately respond to emergency events, whether climate driven, mechanical, as well as over/under irrigation and fertigation measurements and indications of scheduled and non-scheduled activities.

### BRIEF DESCRIPTION OF DRAWINGS

Claimed subject matter is particularly pointed out and distinctly claimed in the concluding portion of the specification. However, both as to organization and/or method of operation, together with objects, features, and/or advantages thereof, it may best be understood by reference to the following detailed description if read with the accompanying drawings in which:

FIG. 1 is a schematic diagram of a system to irrigate a crop field according to a predicted soil, root, and crop demand to create an irrigation schedule, according to an embodiment;

FIG. 2A is a schematic diagram of components of a system for determining a predicted irrigation schedule, according to an embodiment;

FIG. 2B is a schematic diagram of a system for providing a recommended irrigation schedule to a client farmer, according to an embodiment;

FIG. 3 is a flow diagram of a process to determine a predicted irrigation schedule, according to particular embodiments;

FIG. 4 is a flow diagram of a process to train one or more aspects of a neural network model, according to an embodiment;

FIGS. 5A, 5B and 5C are plots showing samples that may be used in inference operations for determining a recommended irrigation schedule, or included in a training set for training parameters of a neural network model, according to an embodiment;

FIG. 6A is a schematic diagram of a generative neural network model, according to one embodiment;

FIG. 6B is a schematic diagram of a generative neural network model, according to another embodiment;

FIG. 7 is a schematic block diagram of an example computing system in accordance with an implementation;

FIG. 8 is a schematic diagram of a neural network formed in “layers”, according to an embodiment; and

FIG. 9 is a flow diagram of an aspect of a training operation, according to an embodiment.

Reference is made in the following detailed description to accompanying drawings, which form a part hereof, wherein like numerals may designate like parts throughout that are corresponding and/or analogous. It will be appreciated that the figures have not necessarily been drawn to scale, such as for simplicity and/or clarity of illustration. For example, dimensions of some aspects may be exaggerated relative to others. Furthermore, structural and/or other changes may be made without departing from claimed subject matter. It should also be noted that directions and/or references, for example, such as up, down, top, bottom, and so on, may be used to facilitate discussion of drawings and are not intended to restrict application of claimed subject matter. Therefore, the following detailed description is not to be taken to limit claimed subject matter and/or equivalents. Further, it is to be understood that other embodiments may be utilized. Also, embodiments have been provided of claimed subject matter and it is noted that, as such, those illustrative embodiments are inventive and/or unconventional; however, claimed subject matter is not limited to embodiments provided primarily for illustrative purposes. Thus, while advantages have been described in connection with illustrative embodiments, claimed subject matter is inventive and/or unconventional for additional reasons not expressly mentioned in connection with those embodiments. In addition, references throughout this specification to “claimed subject matter” refer to subject matter intended to be covered by one or more claims, and are not necessarily intended to refer to a complete claim set, to a particular combination of claim sets (e.g., method claims, apparatus claims, etc.), or to a particular claim.

### DETAILED DESCRIPTION

References throughout this specification to one implementation, an implementation, one embodiment, an embodiment, and/or the like means that a particular feature, structure, characteristic, and/or the like described in relation to a particular implementation and/or embodiment is included in at least one implementation and/or embodiment of claimed subject matter. Thus, appearances of such phrases, for example, in various places throughout this specification are not necessarily intended to refer to the same implementation and/or embodiment or to any one particular implementation and/or embodiment. Furthermore, it is to be understood that particular features, structures, characteristics, and/or the like described are capable of being combined in various ways in one or more implementations and/or embodiments and, therefore, are within intended claim scope. In general, of course, as has always been the case for the specification of a patent application, these and other issues have a potential to vary in a particular context of usage. In other words, throughout the patent application, particular context of description and/or usage provides helpful guidance regarding reasonable inferences to be drawn; however, likewise, “in this context” in general without further qualification refers to the context of the present patent application.

The design of irrigation systems may be improved with the use of technologies that increase the penetration of water into the farmed soil, by reducing the water's surface tension. Some of these technologies may include precisely injected chemical surfactants into the irrigated water column, or introducing energy in the water using very powerful sets of magnets combined with water turbulent devices, or by injecting a measurable electromagnetic signal into a water column, or by conducting electronic energy with a measurable radio frequency (RF) signal that is then propagated by water being treated.

Irrigating with water with a reduced surface tension allows a greater proportion of water to be absorbed in the soil, in measurably less time-resulting in reduced runoff, increased soil water holding capacity, and reduced leaching through the active root zones, and reduced irrigation costs.

As may be observed, the demand for water purchased from a source for irrigating a crop may vary greatly depending on several factors such as time of day, varying growth stages of plants throughout the growing season, and whether the water is a conventional water or water that is treated electronically, magnetically, or chemically to reduce its surface tension. That is, the same crop field may require delivery of “reduced” acre-feet of water for a given irrigation period if the water to be delivered is to be of reduced surface tension.

In one implementation, irrigation of crops in a field may occur according to an irrigation schedule. Such an irrigation schedule may specify, for example, a flow and pressure of the water to be delivered to a crop's field at points in time or periods (e.g., week, day, time of day, etc.). Irrigation schedule may specify a start time, duration and/or flow rate. An irrigation demand for a crop field may be affected by multiple factors such as, for example, variable weather conditions (e.g., temperature, humidity, winds, solar radiation and expected rainfall), evapotranspiration (the demand for water due to the amount of uptake the plant used for growth or lost to evaporation), and varying growth stage-just to provide a few examples of factors that may be considered in determining a precise irrigation schedule. Additionally, characteristics of the electronically generated energy being applied to water for treatment to reduce the water's surface tension may fluctuate due to unintended events and/or conditions that affect energy being propagated by a water column. Also, such a fluctuation of characteristics of the energy may affect a degree to which a surface tension is reduced in treated water, or if energy is present and no energy is conducted in the irrigation system and propagated in such a water column.

Briefly, one embodiment is directed to a method comprising: collecting time-referenced observations and/or measurements from sensors deployed in a crop field, the crop field having been irrigated by a flow of water processed by application of one or more electrical power signals to at least a portion of the flow of water to reduce a surface tension in the flow of water; and computing a recommended irrigation schedule to achieve a target soil moisture based, at least in part, on the time-referenced observations and/or measurements, wherein the time-referenced observations and/or measurements further comprise observations and/or measurements of: water pressure and/or water flow; measurements and/or observations of the one or more electrical power signals and measurements and/or observations of soil moisture in the crop field. By considering observations and/or measurements of electrical power signals used in treating a flow of water, an irrigation schedule may be more precisely determined for achieving an optimized target crop soil moisture for multiple stages in a crop's growth cycle.

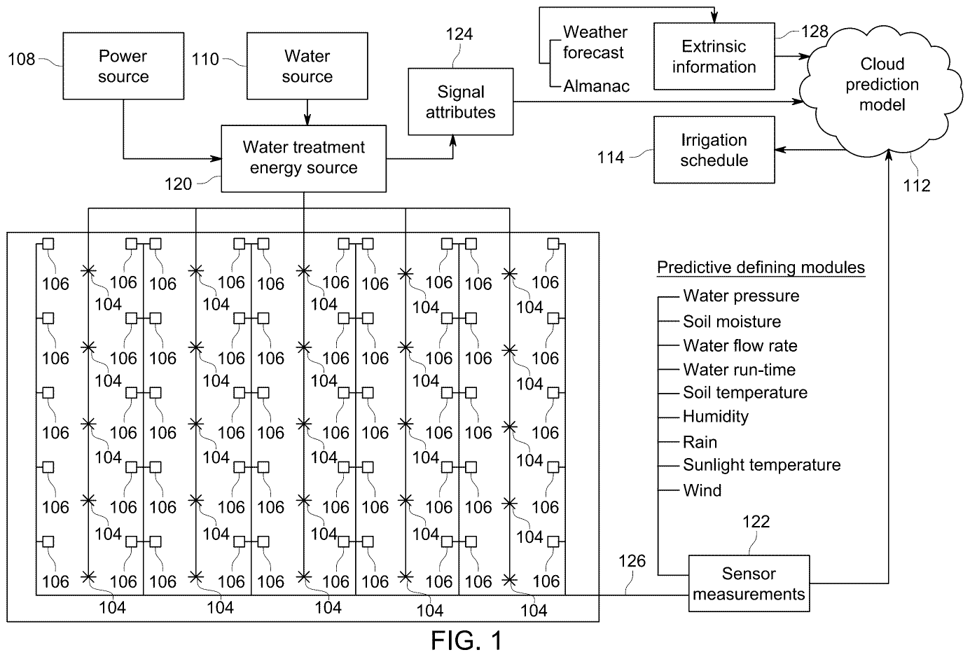

FIG. 1 is a schematic diagram of a system 100 to irrigate a crop field 102 according to a predicted irrigation schedule, according to an embodiment. According to embodiment, crop field 102 may be irrigated with water treated to have a reduced surface tension, which may enable more efficient penetration of irrigation water into the soil of crop field 102. Crop field 102 may be cultivated to grow any one of several types of commercial crops including, for example, almonds, corn, potatoes, lettuce, just to provide a few examples. Water received from water source 110 and treated by water treatment energy source 120 may be distributed through plumbing 116 and nozzles 104 to locations in crop field 102, and ultimate delivery to root systems in soil. Nozzles 104 may implement surface sprinklers, drip irrigation, just to provide a few examples.

According to an embodiment, water delivered from a water source 110 may be treated at water treatment energy source 120 for a reduction in surface tension. For example, water treatment energy source 120 may treat water from water source 110 to reduce surface tension using any one of several techniques such as, for example, introducing a propagating electromagnetic field into the water as it flows through plumbing 116. For example, water delivered to plumbing 116 may pass through pipes of a conductive material prior to delivery to nozzles 104. Water treatment energy source 120 may include load terminals (not shown) connected to such a conductive pipe. Water treatment energy source 120 may then apply a pulsed radio frequency (RF) signal to load terminals on the conductive pipe. The pulsed RF signal may then be applied to water being delivered through the conductive pipe. Here, water surface tension may be reduced to a water surface tension equivalent to water surface tension at up to 90 degrees F. or warmer. Such a technique for treatment of irrigation water may be implemented as shown in U.S. Pat. No. 10,798,887, incorporated herein by reference.

According to an embodiment, a degree to which surface tension in water from water source 110 may be reduced by water treatment energy source 120 may be limited by an availability of electrical power supplied by power source 108. Power source 108 may comprise anyone of several different types of power sources including, for example, utility company power received from a power grid and/or renewable source including such as a battery-backed solar power source, just to provide a couple of examples. In certain scenarios, such an availability of electrical power supplied by power source 108 may be disrupted by any one of several events such as, for example, disconnection of a power line connecting power source 108 from water treatment energy source 120. Such a disruption of power supplied by power source 108 may likewise result in a disruption of water surface tension reduction to be performed at water treatment energy source 120. With a disruption in water surface tension reduction, a higher volume of water may be required to sufficiently irrigate crop field 102.

According to an embodiment, as a component of irrigation schedule 114, cloud prediction model 112 may determine an amount/flow of water to draw from water source 110. In one example implementation, such an amount/flow of water delivered to roots of crop field may optimized based, at least in part, on crop species, soil type/condition or crop maturity, just to provide a few examples. According to an embodiment, cloud prediction model 112 may compute irrigation schedule 114 based, at least in part, on a degree to which water drawn from a water source is to be treated for a reduction in surface tension. In one particular implementation, cloud prediction model 112 may predict and/or recommend an amount of water to be drawn and/or delivered based, at least in part, on multiple different parameters such as, for example, sensor measurements 122, extrinsic information 128 and/or signal attributes 124 received from water treatment energy source 120. In one particular implementation, sensor measurements 122 may be obtained from sensors 106 placed in multiple fixed locations of crop field 102, and may include local measurements for pipe water pressure, ground moisture, air temperature, ground temperature, air humidity, sunlight intensity or wind, just to provide a few examples. These are merely examples of collected observations and/or measurements that may be formatted into modules to enable a precisely predicted optimal irrigation schedule. Extrinsic information 128 may comprise weather forecasts and/or farmer's almanac information. In one particular implementation, cloud prediction model 112 may maintain a history of inputs received in sensor measurements 122 and extrinsic information 128. Cloud prediction model 112 may then compute parameters of irrigation schedule 114 based, at least in part, on such a maintained history.

According to an embodiment, sensor measurements 122 may be organized and/or formatted into “modules” that may be processed by cloud prediction model 112 for training operations or inference/prediction operations. Sensor measurements 122 may be collected from multiple sources. For example, some or all sensor measurements may be collected from sensors 106 over wired and/or wireless communication links. Additionally, some or all of sensor measurements 122 may be provided to cloud prediction model 112 via an application programming interface (API).

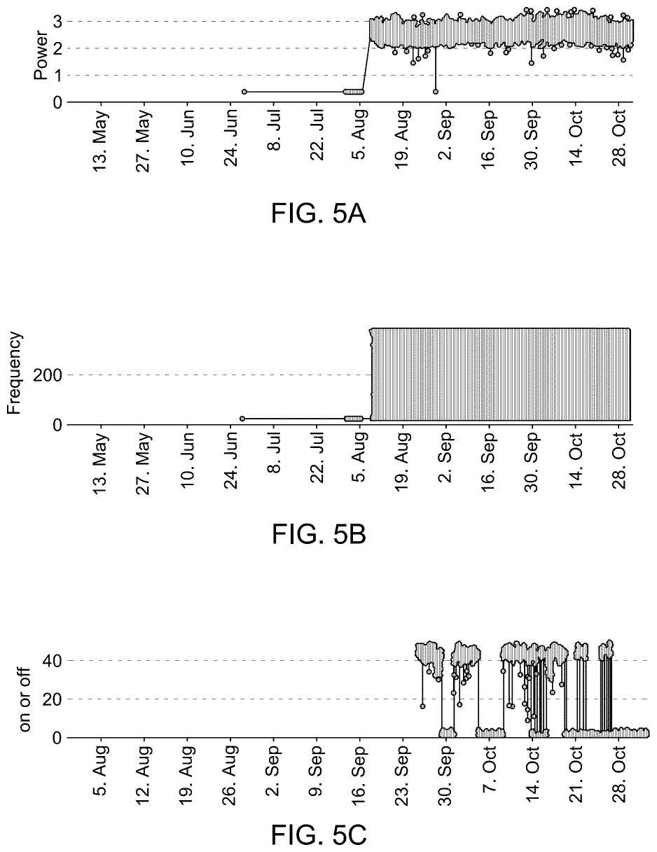

FIGS. 5A, 5B and 5C are plots of sensor measurements that may be used in training operations or inference operations, according to an embodiment. FIG. 5A may comprise a plot of samples of current and/or power of an RF signal applied by water treatment energy source 120, and FIG. 5B may comprise a plot of a frequency of the applied RF signal. FIG. 5C may comprise a plot indicating instances when water is being delivered for irrigation (e.g., through plumbing 116). Here, in training or inference operations, cloud prediction model 112 may be able to determine amounts of water being delivered to irrigate crop field 102 at specific times, and whether such delivered water is treated for reduced surface tension. For example, if the plot of FIG. 5C indicates that a flow of water at an instance is “on” but the plot of FIG. 5A indicates that no current/power is being applied, it may be inferred that water being delivered is untreated for reduced surface tension. Conversely, if the plot of FIG. 5C indicates that a flow of water at an instance is “on” but the plot of FIG. 5A indicates that sufficient current/power is being applied, it may be inferred that water being delivered is treated for reduced surface tension.

In some scenarios, an ability of water treatment energy source 120 to reduce surface tension in water drawn from water source 110 may be suspended and/or disrupted in the event of a disruption in power supplied to water treatment energy source 120 may from power source 108. Such a disruption may occur, for example, as a result of human error and/or negligence. For example, a physical power cable (not shown) connecting power source 108 to water treatment energy source 120 may become disconnected and/or severed by heavy equipment.

In an embodiment, suspension/disruption in an ability of water treatment energy source 120 to reduce surface tension in water drawn from water source 110 may reduce a degree to which water drawn from water source 110 may penetrate soil of crop field 102. In one aspect, signal attributes 124 may indicate whether water treatment energy source 120 is effective at reducing surface tension in water drawn from water source 110 and distributed to nozzles 104 through plumbing 116. In one implementation, signal attributes 124 may comprise and/or be derived from sensing a pulsed RF signal applied to load terminals on a conductive pipe transporting water to/through plumbing 116. For example, signal attributes 124 may comprise an observed/sensed voltage, current, power and/or frequency of a signal applied to load terminals on a conductive pipe. In one aspect, sensing an absence of a pulsed RF signal applied to the load terminals, cloud prediction model 112 may specify in irrigation schedule 114 with an increased amount/flow of water drawn from water source to account for diminished reduction in water surface tension. Such an increase in an amount/flow water may at least in part account for an absence/reduction in surface tension reduction resulting from a removal of power (e.g., from power source 108).

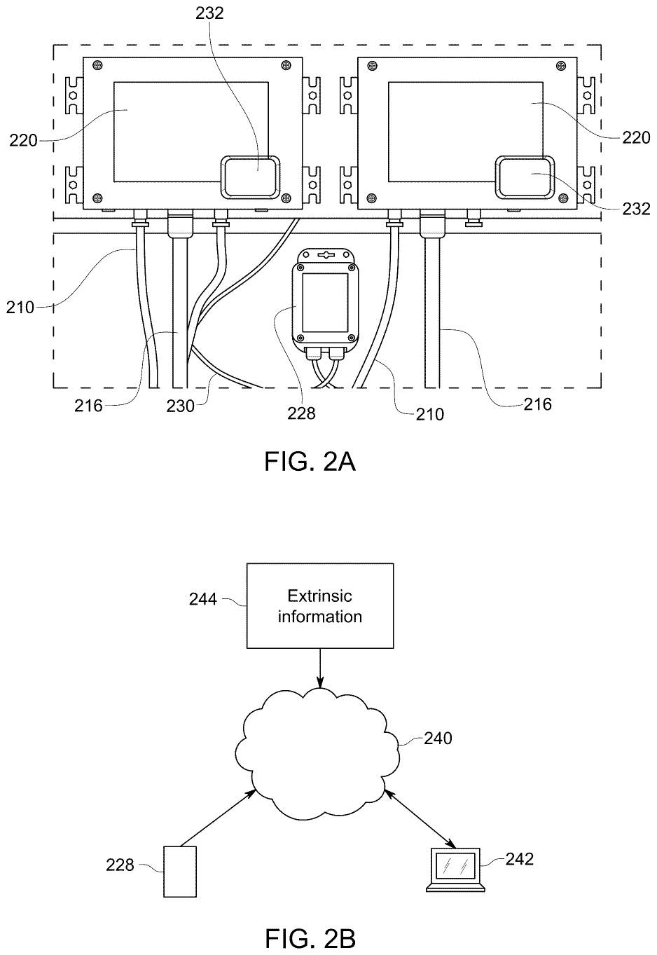

FIG. 2 is a schematic diagram of components of a system for determining an irrigation schedule, according to an embodiment. Water treatment systems 220 may comprise systems to reduce surface tension of water obtained from a water source (e.g., water source 110) through an input conduit (not shown) to deliver treated water through plumbing 216 (e.g., through plumbing 116 for delivery through nozzles 104). Water treatment systems 220 may comprise water treatment systems sold by Flow-Tech Systems, LLC, for example. Here, water treatment systems 220 may apply an RF waveform to water drawn from a water source to be delivered to plumbing 216. The RF waveform may be powered by power cables 210 (e.g., connected to power source 108).

Combiner node 228 may collect measurements and/or observations from multiple sources, and forward the collected measurements and/or observations to one or more remote computing devices (e.g., a cloud computing device, not shown). In particular implementations, combiner node 228 may collect measurements and/or observations from the multiple sources using any one of severely types of wired and wireless communication links including WiFi, Bluetooth, 5G communication, Ethernet, just to provide a few examples. For example, combiner node 228 may collect sensor measurements (e.g., sensor measurements 122) from sensors deployed in a crop field (e.g., sensors 106 deployed in crop field 102). Combiner node 228 may also collect measurements of water pressure in water delivered in plumbing 216 (e.g., from a water pressure sensor) and/or measurements of water flow (e.g., from a water flow meter). Combiner 228 may also collect measurements of a power signal applied for treatment of water to be delivered in plumbing 216. In one example, cables 230 may provide signals indicative of observations of signal attributes (e.g., signal attributes 124). In one embodiment, such observations of signal attributes may be obtained from circuitry to control displays 232 showing power and frequency, for example. In another embodiment, such observations and/or measurements of signal attributes may be measured directly from electrodes connected to conductive pipes.

According to an embodiment, combiner node 228 may tag observations and/or measurements obtained from sensor signals (e.g., sensor measurements 122, measurements of signal attributes 124, water pressure and/or flow measurements) with time stamps (e.g., date and time) to provide “time-referenced” observations and/or measurements comprising primary modules for predictive irrigation scheduling. The time-referenced observations and/or measurements may then be transported to the one or more remote computing devices using any one of several different types of physical communication links.

Such time-referenced measured/observed attributes of electrically injected energy to treat water to be collected at combiner node 228 may include, for example, electrical current, voltage, or frequency of a present RF signal and its existent duration. Such measured/observed attributes obtained from field sensors and irrigation sensors (e.g., sensor measurements 122) collected at combiner node 228 may include, for example, irrigation flow and line pressure rates, scheduled irrigation sets, and soil moisture measurements. According to an embodiment, these measurements and/or observations collected at combiner node 228 may be aggregated at one or more remote computing devices with additional information obtained directly by the one or more remote computing devices. This additional information may include, for example, historical, current season, and real-time weather data, as well as other known farming practices (e.g., extrinsic information 128) that may assist with determining an irrigation schedule that enhances quality and quantity of crops harvested and delivered to market, or used as feed.

According to an embodiment as shown in FIG. 2B, a client farmer may obtained a recommended irrigation schedule for a particular crop field (e.g., crop field 102) through a graphical user interface (GUI) hosted on a computing device 242. Here, one or more remote computing devices may be configured as cloud computing system 240, which may communicate with combiner node 228 and computing device 242 over a communication network using appropriate communication protocols. In one embodiment, combiner node 228 may upload collected measurements and/or observations to cloud computing system 240 via an application programming interface (API), such as an API defined by neatMon, Inc. In a particular implementation, a client farmer may request a recommendation for an irrigation schedule by responding to a prompt presented in the GUI. For example, the client farmer may respond to the prompt by identifying a particular crop field to be irrigated, crops to be irrigated, a point in a growth cycle, target soil moisture (e.g., target percentage of irrigated field capacity), just to provide a few examples.

Based, at least in part, on information provided by the client farmer and additional information collected over time (e.g., uploaded from combiner node 228 and/or extrinsic information 244), cloud computing system 240 may compute a recommended irrigation schedule for a crop field. Such a recommended irrigation schedule may specify particular dates and times when irrigation is to take place, water pressure/flow, irrigation start time and duration, just to provide a few examples of how cloud computing system 240 may express a recommended irrigation schedule to a client farmer. Such a computed recommended irrigation schedule may then be presented to the client farmer in the GUI, provided in an email, text message, etc.

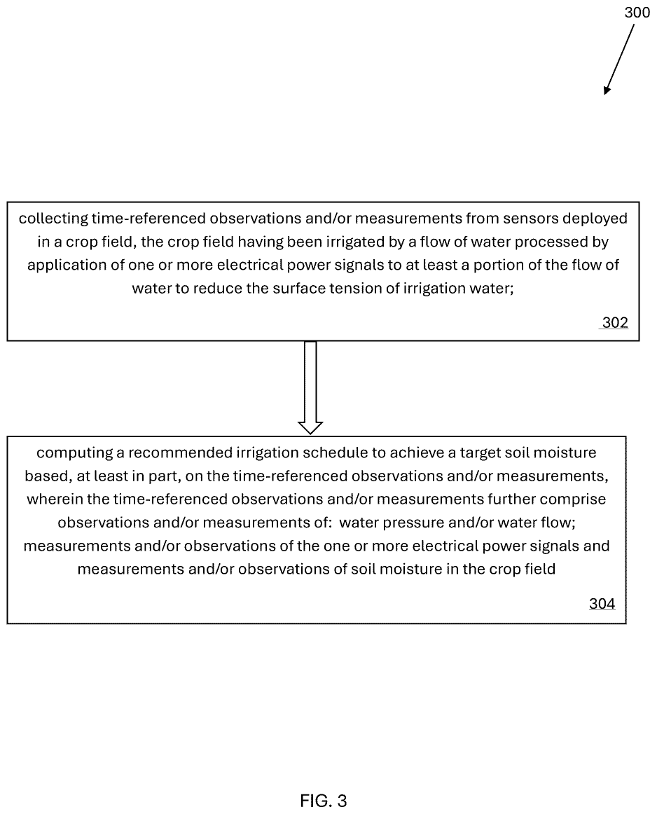

FIG. 3 is a flow diagram of a process 300 for determining an irrigation schedule for a crop field, according to an embodiment. In one particular implementation, all or portions of process 300 may be executed by one or more remote computers to implement cloud computing system 240, for example. Block 302 may comprise obtaining observations and/or measurements collected at combiner node 228. For example, cloud computing system 240 may receive messages from combiner node 228 (e.g., pulled and/or in batches) containing observations and/or measurements collected from sensors 106 deployed in the crop field. As pointed out above, such observations and/or measurements may include observations and/or measurements of ground/soil moisture, air temperature, ground temperature, air humidity, sunlight intensity or wind. Additional observations and/or measurements (e.g., of water flow/pressure and signal attributes of RF waveform applied for reducing surface tension) may be obtained directly from water treatment system 220, for example.

In a particular implementation, observations and/or measurements of water pressure collected at block 302 may be expressed as pounds per square inch. Additionally, in one implementation, observations and/or measurements of signal attributes of an RF waveform applied for reducing water surface tension may be expressed as measured values of current, power, or signal frequency. In another implementation, observations and/or measurements of signal attributes of an RF waveform applied for reducing water surface tension may be expressed as indication of whether the applied RF waveform meets certain threshold conditions (e.g., threshold conditions for current level, power level or frequency, etc.).

Block 304 may comprise computing a recommended irrigation schedule to achieve and/or maintain a target soil moisture based, at least in part, on the time-referenced observations and/or measurements obtained at block 302. Such a recommended irrigation schedule may specify, for example, amounts of water to be drawn from a water source (e.g., water source 110) at particular days, times of day, start time/end time, duration, etc. In one particular implementation, such a target soil moisture may be specified by a client farmer in a GUI (e.g., responsive to a prompt) as a level of moisture to be present in soil or as a percentage of irrigation field capacity. In another particular implementation, a target soil moisture may be specified as a time-varying target soil moisture profile. In another implementation, a target soil moisture may be specified at one or more specific depths beneath a grade line (e.g., 12 inch, 24 inch, 36 inch, etc.).

In addition to using time-referenced observations and/or measurements collected at block 302, block 304 may also compute a recommended irrigation schedule based on other information including, for example, a type of crop to be irrigated, point in growth cycle, extrinsic information 244, etc. Additionally, block 304 may determine a recommended irrigation schedule further based, at least in part, on 1) client provided data modules (e.g., containing sensor measurements 122), 2) client provided crop-water-need values and/or 3) weather forecast/almanac (e.g., in extrinsic information 128 and/or 244), just to provide a few examples.

According to an embodiment, cloud prediction model 112 may employ a machine-learning system to generate and/or predict aspects of irrigation schedule 114. In one particular implementation, for a particular customer (e.g., farmer) for a particular crop field (e.g., crop field 102), parameters of one or more neural networks may be trained to generate a recommended irrigation schedule. In one embodiment, a history of observations and/or conditions for a particular farmer client and/or particular crop field (e.g., time-referenced observations and/or measurements collected at combiner node 228, time-referenced observations and/or measurements of signal attributes of an RF waveform applied for reducing water surface tension at water treatment system 220, time-referenced observations and/or measurements of water pressure/waterflow of water delivered to plumbing 216, etc.) may be collected over multiple growing seasons, multiple crop types, etc. for use in training a machine-learning model.

In one embodiment, observations and/or conditions for a particular farmer client and/or particular crop field collected over multiple growing seasons may be used to train a proprietary machine learning model, such as a machine learning model implemented by neatMon, Inc., for example. Here, such a machine learning model may be trained to provide a recommended irrigation schedule for the particular farmer client and/or particular crop field based on a target soil moisture to implement block 304.

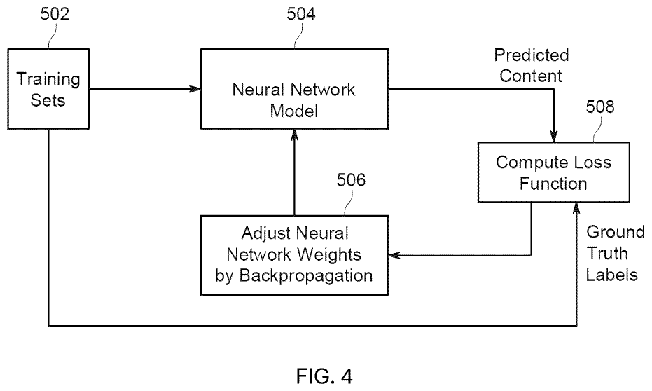

According to an embodiment, operations to train a neural network model (e.g., for implementation in block 304 (FIG. 3) cloud prediction model 112 (FIG. 1)) to generate aspects of an irrigation schedule may be executed according to process 500 shown in FIG. 4. In one example, a neural network model 504 in a training operation may be trained to compute/predict parameters (e.g., amounts of water to be applied in irrigation of a crop field on particular days, times of day, etc. as part of an irrigation schedule). On training epochs/iterations, such computed/predicted parameters may be compared with paired ground truth labels in training sets 502 for computation of a loss function at block 508. Neural network weights of neural network model 504 may then be adjusted at block 506 using backpropagation, for example, for execution of neural network model 504 in a subsequent training epoch/iteration. In one embodiment, training sets 502 may be obtained from observations as presented in plots shown in FIGS. 5A, 5B and 5C, for example.

In one particular implementation, block 508 may compute a loss function according to one or more particular formulations such as a least squares error formulation. On any particular training epoch/iteration i, block 508 may compute loss value(s) Cᵢ (yᵢ, ŷᵢ) according to expression (1) as follows:

$${C}_{i} ( {y}_{i} , {\hat{y}}_{i} ) = L ( {y}_{i} , {\hat{y}}_{i} ) , \qquad ( 1 )$$

where:
yᵢ is a ground truth label obtained from training sets 502 for training epoch/iteration i;
ŷᵢ is an output computed by neural network model 504 based, at least in part, on input value(s) obtained from training sets 502 for training epoch/iteration i;
L is a suitable loss function.

Block 506 may then adjust/update neural network weights of neural network model 504 in a training epoch/iteration i based, at least in part, on a computed value for Cᵢ (yᵢ, ŷᵢ).

In one example, neural network model 504 may be trained to generate/predict parameters relating to an irrigation schedule. Here, training sets 502 applied in such a subsequent training operation may comprise time-referenced observations and/or events as inputs to neural network model 504 on training epochs/iterations, paired with irrigation events as ground truth labels yᵢ. Such time-referenced observations and/or events as inputs to neural network model 504 may comprise, for example, observations obtained from sensors 106 including soil moisture observations (e.g., at multiple soil depths), water pressure/flow in plumbing 116, air temperature, air humidity, wind, sunlight intensity, just to provide a few examples. Such time-referenced observations and/or events may comprise time-referenced signal attributes 124, which may include observations of characteristics of an RF signal (e.g., current, power, frequency, etc.) applied by water treatment energy source 120 to water drawn from water source 110 and delivered by plumbing 116.

In one implementation, parameters of neural network model 504 may be trained to compute a particular predicted “irrigation event” to result in a particular target soil moisture level for a particular crop field and/or client farmer. Such a predicted irrigation event may comprise, for example, a flow of water (treated and/or untreated) to be applied to a particular crop field on a particular day, start time, duration and/or end time. Such an irrigation event may be among one or more such irrigation events set forth in a recommended irrigation schedule, for example. In training operations, for example, ground truth labels yᵢ provided to block 508 may comprise collected time-referenced observations and/or measurements of soil moisture and/or soil nutrients in the particular crop field. In one embodiment, predictions ŷᵢ may comprise predicted soil moisture levels at different times for a given history of observations and/or conditions.

In another embodiment, predictions ŷᵢ may comprise time-referenced target soil moisture levels for which irrigation events are to be computed to achieve such time-referenced moisture levels. Here, corresponding time-referenced observations and/or measurements of soil moisture and/or soil nutrients in the particular crop field following application of such irrigation events to crop field may provide ground truth labels yᵢ.

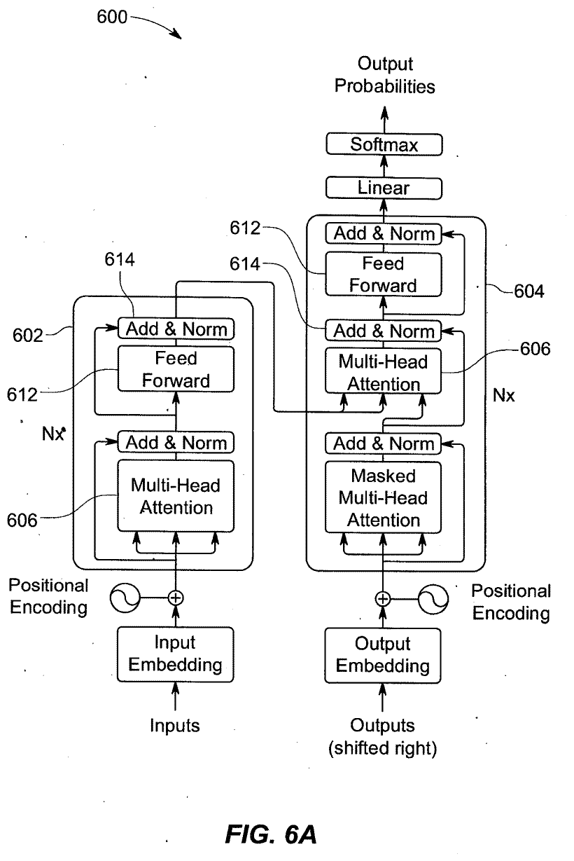

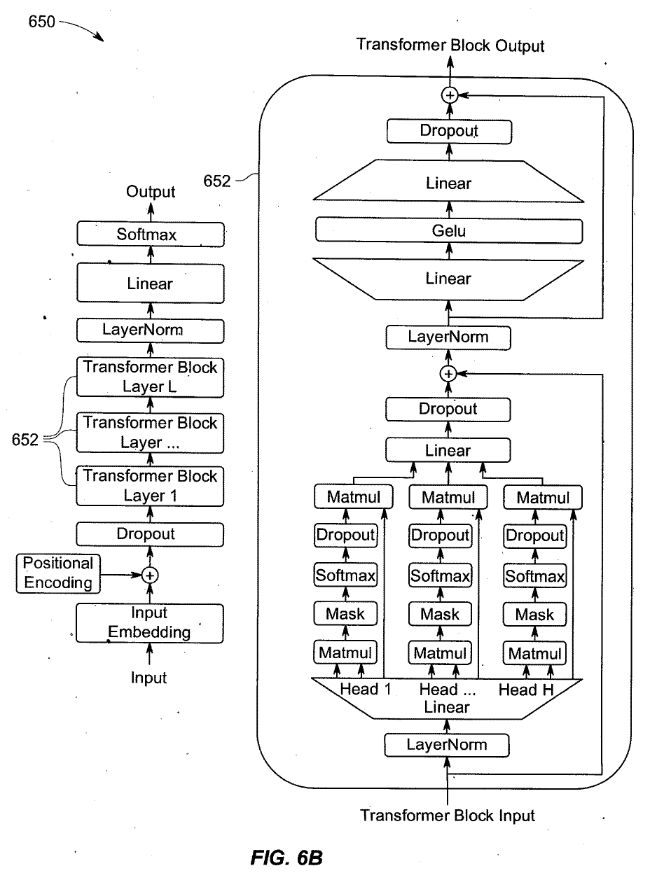

According to another embodiment, a neural network model to implement cloud prediction model 112 (e.g., neural network model 504) may be configured as a natural language processing (NLP) model, such as LLMs powered by versions of models such as LongT5, MPT, and Llama2, just to provide a few examples. As one example of such an NLP model, FIG. 6A is a schematic diagram of a neural network model 600 such as an implementation of generative pretrained model, such as GPT, for example. As another example of such an NLP model, FIG. 6B is a schematic diagram of another embodiment of generative neural network model 650 as an implementation of a generative pretrained model using a series of transformers 652. In one implementation, inputs generative neural network model 600 and/or 650 may comprise a series of words that are preprocessed (e.g., converted to numbers or other input vectors) and provided in sequence to generate output probabilities of a subsequent word. Once the subsequent word is determined, the subsequent word may be combined with the input so that the next subsequent word may be determined, causing transformers to repeatedly predict a next word in a response to a prompt. In one implementation, an input sequence may be fixed at some value, such as 2048 words, and extra positions at the beginning may be padded with zeros. An output may similarly comprise an array of possible outcomes with associated probabilities, such that the most probable subsequent word may be selected as the next word in the response or output.

Because input vectors in this particular example may indicate only a single word and comprise many more zeros than ones (e.g., GPT has a vocabulary of over 50,000 input words and associated vectors), the input vectors may be embedded or encoded into a smaller multidimensional space at an input embedding element. In generative neural network model 600 in particular, the position of each resulting token in a sequence of inputs may be encoded and provided to a multi-head attention element 606 operable to predict a degree to which an input token is likely to impact an output. Feed-forward blocks 612 may each comprise a multi-layer neural network, operable to learn over time to predict the next word in a sequence. An add & norm block 614 may combine and normalize outputs of multiple previous blocks.

In the context of the present patent application, the term “connection,” the term “component” and/or similar terms are intended to be physical, but are not necessarily always tangible. Whether or not these terms refer to tangible subject matter, thus, may vary in a particular context of usage. As an example, a tangible connection and/or tangible connection path may be made, such as by a tangible, electrical connection, such as an electrically conductive path comprising metal or other conductor, that is able to conduct electrical current between two tangible components. Likewise, a tangible connection path may be at least partially affected and/or controlled, such that, as is typical, a tangible connection path may be open or closed, at times resulting from influence of one or more externally derived signals, such as external currents and/or voltages, such as for an electrical switch. Non-limiting illustrations of an electrical switch include a transistor, a diode, etc. However, a “connection” and/or “component,” in a particular context of usage, likewise, although physical, can also be non-tangible, such as a connection between a client and a server over a network, particularly a wireless network, which generally refers to the ability for the client and server to transmit, receive, and/or exchange communications, as discussed in more detail later.

In a particular context of usage, such as a particular context in which tangible components are being discussed, therefore, the terms “coupled” and “connected” are used in a manner so that the terms are not synonymous. Similar terms may also be used in a manner in which a similar intention is exhibited. Thus, “connected” is used to indicate that two or more tangible components and/or the like, for example, are tangibly in direct physical contact. Thus, using the previous example, two tangible components that are electrically connected are physically connected via a tangible electrical connection, as previously discussed. However, “coupled,” is used to mean that potentially two or more tangible components are tangibly in direct physical contact. Nonetheless, “coupled” is also used to mean that two or more tangible components and/or the like are not necessarily tangibly in direct physical contact, but are able to co-operate, liaise, and/or interact, such as, for example, by being “optically coupled.” Likewise, the term “coupled” is also understood to mean indirectly connected. It is further noted, in the context of the present patent application, since memory, such as a memory component and/or memory states, is intended to be non-transitory, the term physical, at least if used in relation to memory necessarily implies that such memory components and/or memory states, continuing with the example, are tangible.

Unless otherwise indicated, in the context of the present patent application, the term “or” if used to associate a list, such as A, B, or C, is intended to mean A, B, and C, here used in the inclusive sense, as well as A, B, or C, here used in the exclusive sense. With this understanding, “and” is used in the inclusive sense and intended to mean A, B, and C; whereas “and/or” can be used in an abundance of caution to make clear that all of the foregoing meanings are intended, although such usage is not required. In addition, the term “one or more” and/or similar terms is used to describe any feature, structure, characteristic, and/or the like in the singular, “and/or” is also used to describe a plurality and/or some other combination of features, structures, characteristics, and/or the like. Likewise, the term “based on” and/or similar terms are understood as not necessarily intending to convey an exhaustive list of factors, but to allow for existence of additional factors not necessarily expressly described.

The terms “correspond”, “reference”, “associate”, and/or similar terms relate to signals, signal samples and/or states, e.g., components of a signal measurement vector, which may be stored in memory and/or employed with operations to generate results, depending, at least in part, on the above-mentioned, signal samples and/or signal sample states. For example, a signal sample measurement vector may be stored in a memory location and further referenced wherein such a reference may be embodied and/or described as a stored relationship. A stored relationship may be employed by associating (e.g., relating) one or more memory addresses to one or more another memory addresses, for example, and may facilitate an operation, involving, at least in part, a combination of signal samples and/or states stored in memory, such as for processing by a processor and/or similar device, for example. Thus, in a particular context, “associating,” “referencing,” and/or “corresponding” may, for example, refer to an executable process of accessing memory contents of two or more memory locations, e.g., to facilitate execution of one or more operations among signal samples and/or states, wherein one or more results of the one or more operations may likewise be employed for additional processing, such as in other operations, or may be stored in the same or other memory locations, as may, for example, be directed by executable instructions. Furthermore, terms “fetching” and “reading” or “storing” and “writing” are to be understood as interchangeable terms for the respective operations, e.g., a result may be fetched (or read) from a memory location; likewise, a result may be stored in (or written to) a memory location.

It is further noted that the terms “type” and/or “like,” if used, such as with a feature, structure, characteristic, and/or the like, using “optical” or “electrical” as simple examples, means at least partially of and/or relating to the feature, structure, characteristic, and/or the like in such a way that presence of minor variations, even variations that might otherwise not be considered fully consistent with the feature, structure, characteristic, and/or the like, do not in general prevent the feature, structure, characteristic, and/or the like from being of a “type” and/or being “like,” (such as being an “optical-type” or being “optical-like,” for example) if the minor variations are sufficiently minor so that the feature, structure, characteristic, and/or the like would still be considered to be substantially present with such variations also present. Thus, continuing with this example, the terms optical-type and/or optical-like properties are necessarily intended to include optical properties. Likewise, the terms electrical-type and/or electrical-like properties, as another example, are necessarily intended to include electrical properties. It should be noted that the specification of the present patent application merely provides one or more illustrative examples and claimed subject matter is intended to not be limited to one or more illustrative examples; however, again, as has always been the case with respect to the specification of a patent application, particular context of description and/or usage provides helpful guidance regarding reasonable inferences to be drawn.

With advances in technology, it has become more typical to employ distributed computing and/or communication approaches in which portions of a process, such as signal processing of signal samples, for example, may be allocated among various devices, including one or more client devices and/or one or more server devices, via a computing and/or communications network, for example. A network may comprise two or more devices, such as network devices and/or computing devices, and/or may couple devices, such as network devices and/or computing devices, so that signal communications, such as in the form of signal packets and/or signal frames (e.g., comprising one or more signal samples), for example, may be exchanged, such as between a server device and/or a client device, as well as other types of devices, including between wired and/or wireless devices coupled via a wired and/or wireless network, for example.

An example of a distributed computing system comprises the so-called Hadoop distributed computing system, which employs a map-reduce type of architecture. In the context of the present patent application, the terms map-reduce architecture and/or similar terms are intended to refer to a distributed computing system implementation and/or embodiment for processing and/or for generating larger sets of signal samples employing map and/or reduce operations for a parallel, distributed process performed over a network of devices. A map operation and/or similar terms refer to processing of signals (e.g., signal samples) to generate one or more key-value pairs and to distribute the one or more pairs to one or more devices of the system (e.g., network). A reduce operation and/or similar terms refer to processing of signals (e.g., signal samples) via a summary operation (e.g., such as counting the number of students in a queue, yielding name frequencies, etc.). A system may employ such an architecture, such as by marshaling distributed server devices, executing various tasks in parallel, and/or managing communications, such as signal transfers, between various parts of the system (e.g., network), in an embodiment. As mentioned, one non-limiting, but well-known, example comprises the Hadoop distributed computing system. It refers to an open source implementation and/or embodiment of a map-reduce type architecture (available from the Apache Software Foundation, 1901 Munsey Drive, Forrest Hill, MD, 21050-2747), but may include other aspects, such as the Hadoop distributed file system (HDFS) (available from the Apache Software Foundation, 1901 Munsey Drive, Forrest Hill, MD, 21050-2747). In general, therefore, “Hadoop” and/or similar terms (e.g., “Hadoop-type,” etc.) refer to an implementation and/or embodiment of a scheduler for executing larger processing jobs using a map-reduce architecture over a distributed system. Furthermore, in the context of the present patent application, use of the term “Hadoop” is intended to include versions, presently known and/or to be later developed.

In the context of the present patent application, the term network device refers to any device capable of communicating via and/or as part of a network and may comprise a computing device. While network devices may be capable of communicating signals (e.g., signal packets and/or frames), such as via a wired and/or wireless network, they may also be capable of performing operations associated with a computing device, such as arithmetic and/or logic operations, processing and/or storing operations (e.g., storing signal samples), such as in memory as tangible, physical memory states, and/or may, for example, operate as a server device and/or a client device in various embodiments. Network devices capable of operating as a server device, a client device and/or otherwise, may include, as examples, dedicated rack-mounted servers, desktop computers, laptop computers, set top boxes, tablets, netbooks, smart phones, wearable devices, integrated devices combining two or more features of the foregoing devices, and/or the like, or any combination thereof. As mentioned, signal packets and/or frames, for example, may be exchanged, such as between a server device and/or a client device, as well as other types of devices, including between wired and/or wireless devices coupled via a wired and/or wireless network, for example, or any combination thereof. It is noted that the terms, server, server device, server computing device, server computing platform and/or similar terms are used interchangeably. Similarly, the terms client, client device, client computing device, client computing platform and/or similar terms are also used interchangeably. While in some instances, for ease of description, these terms may be used in the singular, such as by referring to a “client device” or a “server device,” the description is intended to encompass one or more client devices and/or one or more server devices, as appropriate. Along similar lines, references to a “database” are understood to mean, one or more databases and/or portions thereof, as appropriate.

It should be understood that for ease of description, a network device (also referred to as a networking device) may be embodied and/or described in terms of a computing device and vice-versa. However, it should further be understood that this description should in no way be construed so that claimed subject matter is limited to one embodiment, such as only a computing device and/or only a network device, but, instead, may be embodied as a variety of devices or combinations thereof, including, for example, one or more illustrative examples.

A network may also include now known, and/or to be later developed arrangements, derivatives, and/or improvements, including, for example, past, present and/or future mass storage, such as network attached storage (NAS), a storage area network (SAN), and/or other forms of device readable media, for example. A network may include a portion of the Internet, one or more local area networks (LANs), one or more wide area networks (WANs), wire-line type connections, wireless type connections, other connections, or any combination thereof. Thus, a network may be worldwide in scope and/or extent. Likewise, sub-networks, such as may employ differing architectures and/or may be substantially compliant and/or substantially compatible with differing protocols, such as network computing and/or communications protocols (e.g., network protocols), may interoperate within a larger network.

The Internet refers to a decentralized global network of interoperable networks that comply with the Internet Protocol (IP). It is noted that there are several versions of the Internet Protocol. The term Internet Protocol, IP, and/or similar terms are intended to refer to any version, now known and/or to be later developed. The Internet includes local area networks (LANs), wide area networks (WANs), wireless networks, and/or long haul public networks that, for example, may allow signal packets and/or frames to be communicated between LANs. The term World Wide Web (WWW or Web) and/or similar terms may also be used, although it refers to a part of the Internet that complies with the Hypertext Transfer Protocol (HTTP). For example, network devices may engage in an HTTP session through an exchange of appropriately substantially compatible and/or substantially compliant signal packets and/or frames. It is noted that there are several versions of the Hypertext Transfer Protocol. The term Hypertext Transfer Protocol, HTTP, and/or similar terms are intended to refer to any version, now known and/or to be later developed. It is likewise noted that in various places in this document substitution of the term Internet with the term World Wide Web (“Web”) may be made without a significant departure in meaning and may, therefore, also be understood in that manner if the statement would remain correct with such a substitution.

The term electronic file and/or the term electronic document are used throughout this document to refer to a set of stored memory states and/or a set of physical signals associated in a manner so as to thereby at least logically form a file (e.g., electronic) and/or an electronic document. That is, it is not meant to implicitly reference a particular syntax, format and/or approach used, for example, with respect to a set of associated memory states and/or a set of associated physical signals. If a particular type of file storage format and/or syntax, for example, is intended, it is referenced expressly. It is further noted an association of memory states, for example, may be in a logical sense and not necessarily in a tangible, physical sense. Thus, although signal and/or state components of a file and/or an electronic document, for example, are to be associated logically, storage thereof, for example, may reside in one or more different places in a tangible, physical memory, in an embodiment.

A Hyper Text Markup Language (“HTML”), for example, may be utilized to specify digital content and/or to specify a format thereof, such as in the form of an electronic file and/or an electronic document, such as a Web page, Web site, etc., for example. An Extensible Markup Language (“XML”) may also be utilized to specify digital content and/or to specify a format thereof, such as in the form of an electronic file and/or an electronic document, such as a Web page, Web site, etc., in an embodiment. Of course, HTML and/or XML are merely examples of “markup” languages, provided as non-limiting illustrations. Furthermore, HTML and/or XML are intended to refer to any version, now known and/or to be later developed, of these languages. Likewise, claimed subject matter are not intended to be limited to examples provided as illustrations, of course.

In the context of the present patent application, the terms “entry,” “electronic entry,” “document,” “electronic document,” “content,”, “digital content,” “item,” and/or similar terms are meant to refer to signals and/or states in a physical format, such as a digital signal and/or digital state format, e.g., that may be perceived by a user if displayed, played, tactilely generated, etc. and/or otherwise executed by a device, such as a digital device, including, for example, a computing device, but otherwise might not necessarily be readily perceivable by humans (e.g., if in a digital format). Likewise, in the context of the present patent application, digital content provided to a user in a form so that the user is able to readily perceive the underlying content itself (e.g., content presented in a form consumable by a human, such as hearing audio, feeling tactile sensations and/or seeing images, as examples) is referred to, with respect to the user, as “consuming” digital content, “consumption” of digital content, “consumable” digital content and/or similar terms. In another embodiment, an electronic document, electronic content and/or digital content may comprise text, audio and/or image content formatted to be processed by a neural network model, or text, audio and/or image content generated by a neural network model. For one or more embodiments, an electronic document and/or an electronic file may comprise a Web page of code (e.g., computer instructions) in a markup language executed or to be executed by a computing and/or networking device, for example. In another embodiment, an electronic document and/or electronic file may comprise a portion and/or a region of a Web page. However, claimed subject matter is not intended to be limited in these respects.

Also, for one or more embodiments, an electronic document and/or electronic file may comprise a number of components. As previously indicated, in the context of the present patent application, a component is physical, but is not necessarily tangible. As an example, components with reference to an electronic document and/or electronic file, in one or more embodiments, may comprise text, for example, in the form of physical signals and/or physical states (e.g., capable of being physically displayed). Typically, memory states, for example, comprise tangible components, whereas physical signals are not necessarily tangible, although signals may become (e.g., be made) tangible, such as if appearing on a tangible display, for example, as is not uncommon. Also, for one or more embodiments, components with reference to an electronic document and/or electronic file may comprise a graphical object, such as, for example, an image, such as a digital image, and/or sub-objects, including attributes thereof, which, again, comprise physical signals and/or physical states (e.g., capable of being tangibly displayed). In an embodiment, digital content may comprise, for example, text, images, audio, video, and/or other types of electronic documents and/or electronic files, including portions thereof, for example.

Also, in the context of the present patent application, the term parameters (e.g., one or more parameters) refer to material descriptive of a collection of signal samples, such as one or more electronic documents and/or electronic files, and exist in the form of physical signals and/or physical states, such as memory states. For example, one or more parameters, such as referring to an electronic document and/or an electronic file comprising an image, may include, as examples, time of day at which an image was captured, latitude and longitude of an image capture device, such as a camera, for example, etc. In another example, one or more parameters relevant to digital content, such as digital content comprising a technical article, as an example, may include one or more authors, for example. Claimed subject matter is intended to embrace meaningful, descriptive parameters in any format, so long as the one or more parameters comprise physical signals and/or states, which may include, as parameter examples, collection name (e.g., electronic file and/or electronic document identifier name), technique of creation, purpose of creation, time and date of creation, logical path if stored, coding formats (e.g., type of computer instructions, such as a markup language) and/or standards and/or specifications used so as to be protocol compliant (e.g., meaning substantially compliant and/or substantially compatible) for one or more uses, and so forth.

Signal packet communications and/or signal frame communications, also referred to as signal packet transmissions and/or signal frame transmissions (or merely “signal packets” or “signal frames”), may be communicated between nodes of a network, where a node may comprise one or more network devices and/or one or more computing devices, for example. As an illustrative example, but without limitation, a node may comprise one or more sites employing a local network address, such as in a local network address space. Likewise, a device, such as a network device and/or a computing device, may be associated with that node. It is also noted that in the context of this patent application, the term “transmission” is intended as another term for a type of signal communication that may occur in any one of a variety of situations. Thus, it is not intended to imply a particular directionality of communication and/or a particular initiating end of a communication path for the “transmission” communication. For example, the mere use of the term in and of itself is not intended, in the context of the present patent application, to have particular implications with respect to the one or more signals being communicated, such as, for example, whether the signals are being communicated “to” a particular device, whether the signals are being communicated “from” a particular device, and/or regarding which end of a communication path may be initiating communication, such as, for example, in a “push type” of signal transfer or in a “pull type” of signal transfer. In the context of the present patent application, push and/or pull type signal transfers are distinguished by which end of a communications path initiates signal transfer.

Thus, a signal packet and/or frame may, as an example, be communicated via a communication channel and/or a communication path, such as comprising a portion of the Internet and/or the Web, from a site via an access node coupled to the Internet or vice-versa. Likewise, a signal packet and/or frame may be forwarded via network nodes to a target site coupled to a local network, for example. A signal packet and/or frame communicated via the Internet and/or the Web, for example, may be routed via a path, such as either being “pushed” or “pulled,” comprising one or more gateways, servers, etc. that may, for example, route a signal packet and/or frame, such as, for example, substantially in accordance with a target and/or destination address and availability of a network path of network nodes to the target and/or destination address. Although the Internet and/or the Web comprise a network of interoperable networks, not all of those interoperable networks are necessarily available and/or accessible to the public.

A network and/or sub-network, in an embodiment, may communicate via signal packets and/or signal frames, such as via participating digital devices and may be substantially compliant and/or substantially compatible with, but is not limited to, now known and/or to be developed, versions of any of the following network protocol stacks: ARCNET, AppleTalk, ATM, Bluetooth, DECnet, Ethernet, FDDI, Frame Relay, HIPPI, IEEE 1394, IEEE 802.11, IEEE-488, Internet Protocol Suite, IPX, Myrinet, OSI Protocol Suite, QsNet, RS-232, SPX, System Network Architecture, Token Ring, USB, and/or X.25. A network and/or sub-network may employ, for example, a version, now known and/or later to be developed, of the following: TCP/IP, UDP, DECnet, NetBEUI, IPX, AppleTalk and/or the like. Versions of the Internet Protocol (IP) may include IPv4, IPv6, and/or other later to be developed versions.

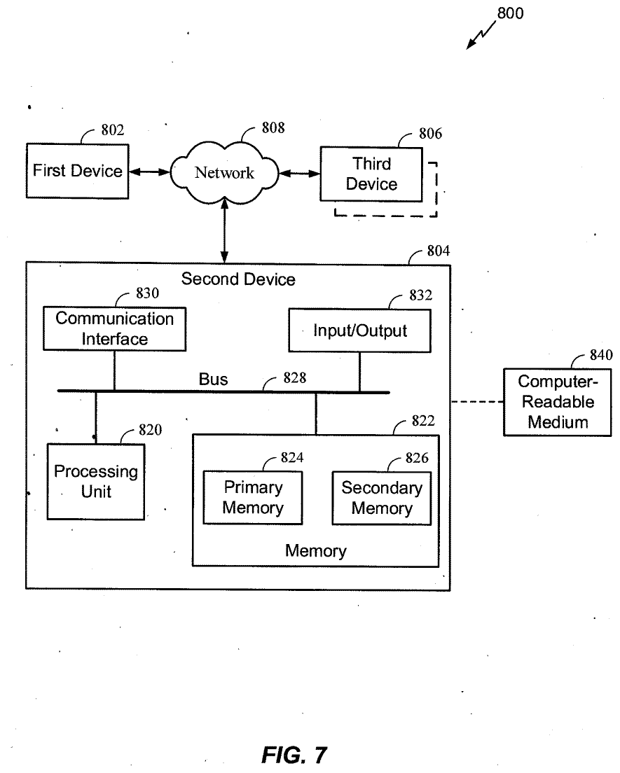

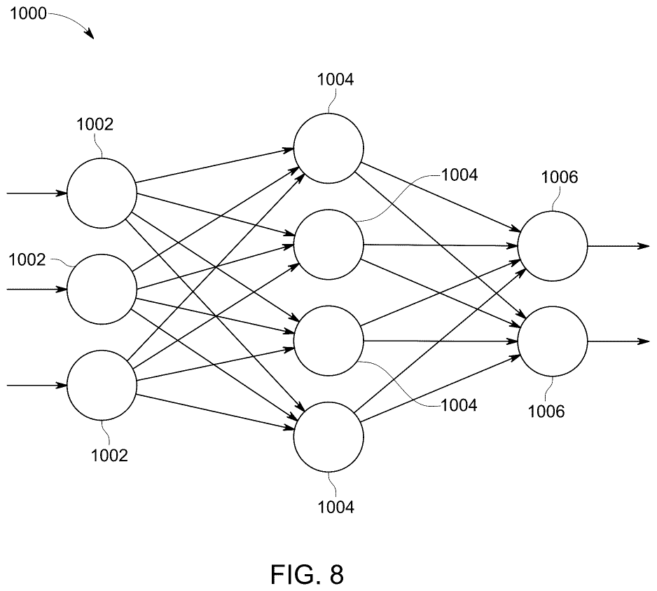

In one example embodiment, as shown in FIG. 7, a system embodiment may comprise a local network (e.g., device 804 and medium 840) and/or another type of network, such as a computing and/or communications network. For purposes of illustration, therefore, FIG. 7 shows an embodiment 800 of a system that may be employed to implement either type or both types of networks. Network 808 may comprise one or more network connections, links, processes, services, applications, and/or resources to facilitate and/or support communications, such as an exchange of communication signals, for example, between a computing device, such as 802, and another computing device, such as 806, which may, for example, comprise one or more client computing devices and/or one or more server computing device. By way of example, but not limitation, network 808 may comprise wireless and/or wired communication links, telephone and/or telecommunications systems, Wi-Fi networks, WiMAX networks, the Internet, a local area network (LAN), a wide area network (WAN), or any combinations thereof.

Example devices in FIG. 7 may comprise features, for example, of a client computing device (e.g., computing device 242) and/or a server computing device (e.g., cloud computing system 240), in an embodiment. It is further noted that the term computing device, in general, whether employed as a client and/or as a server, or otherwise, refers at least to a processor and a memory connected by a communication bus. A “processor,” for example, is understood to connote a specific structure such as a central processing unit (CPU) of a computing device which may include a control unit and an execution unit. In an aspect, a processor may comprise a device that fetches, interprets and executes instructions to process input signals to provide output signals. As such, in the context of the present patent application at least, computing device and/or processor are understood to refer to sufficient structure within the meaning of 35 USC § 112 (f) so that it is specifically intended that 35 USC § 112 (f) not be implicated by use of the term “computing device,” “processor” and/or similar terms; however, if it is determined, for some reason not immediately apparent, that the foregoing understanding cannot stand and that 35 USC § 112 (f), therefore, necessarily is implicated by the use of the term “computing device,” “processor” and/or similar terms, then, it is intended, pursuant to that statutory section, that corresponding structure, material and/or acts for performing one or more functions be understood and be interpreted to be described at least in FIGS. 2A, 2B, 3, 4, 6A and 6B, and in the text associated with the foregoing figure(s) of the present patent application.

Referring now to FIG. 7, in an embodiment, first and third devices 802 and 806 may be capable of rendering a graphical user interface (GUI) (e.g., including a pointer device) for a network device and/or a computing device, for example, so that a user-operator may engage in system use. Computing device 804 may potentially serve a similar function in this illustration. Likewise, in FIG. 7, computing device 802 (‘first device’ in figure) may interface with computing device 804 (‘second device’ in figure), which may, for example, also comprise features of a client computing device and/or a server computing device, in an embodiment. Processor (e.g., processing device) 820 and memory 822, which may comprise primary memory 824 and secondary memory 826, may communicate by way of a communication bus 815, for example. The term “computing device,” in the context of the present patent application, refers to a system and/or a device, such as a computing apparatus, that includes a capability to process (e.g., perform computations) and/or store digital content, such as electronic files, electronic documents, measurements, text, images, video, audio, etc. in the form of signals and/or states. Thus, a computing device, in the context of the present patent application, may comprise hardware, software, firmware, or any combination thereof (other than software per se). Computing device 804, as depicted in FIG. 7, is merely one example, and claimed subject matter is not limited in scope to this particular example.

For one or more embodiments, a device, such as a computing device and/or networking device, may comprise, for example, any of a wide range of digital electronic devices, including, but not limited to, desktop and/or notebook computers, high-definition televisions, digital versatile disc (DVD) and/or other optical disc players and/or recorders, game consoles, satellite television receivers, cellular telephones, tablet devices, wearable devices, personal digital assistants, mobile audio and/or video playback and/or recording devices, Internet of Things (IOT) type devices, or any combination of the foregoing. Further, unless specifically stated otherwise, a process as described, such as with reference to flow diagrams and/or otherwise, may also be executed and/or affected, in whole or in part, by a computing device and/or a network device. A device, such as a computing device and/or network device, may vary in terms of capabilities and/or features. Claimed subject matter is intended to cover a wide range of potential variations. For example, a device may include a numeric keypad and/or other display of limited functionality, such as a monochrome liquid crystal display (LCD) for displaying text, for example. In contrast, however, as another example, a web-enabled device may include a physical and/or a virtual keyboard, mass storage, one or more accelerometers, one or more gyroscopes, global positioning system (GPS) and/or other location-identifying type capability, and/or a display with a higher degree of functionality, such as a touch-sensitive color 2D or 3D display, for example.

As suggested previously, communications between a computing device and/or a network device and a wireless network may be in accordance with known and/or to be developed network protocols including, for example, global system for mobile communications (GSM), enhanced data rate for GSM evolution (EDGE), 802.11b/g/n/h, etc., and/or worldwide interoperability for microwave access (WiMAX). A computing device and/or a networking device may also have a subscriber identity module (SIM) card, which, for example, may comprise a detachable or embedded smart card that is able to store subscription content of a user, and/or is also able to store a contact list. It is noted, however, that a SIM card may also be electronic, meaning that may simply be stored in a particular location in memory of the computing and/or networking device. A user may own the computing device and/or network device or may otherwise be a user, such as a primary user, for example. A device may be assigned an address by a wireless network operator, a wired network operator, and/or an Internet Service Provider (ISP). For example, an address may comprise a domestic or international telephone number, an Internet Protocol (IP) address, and/or one or more other identifiers. In other embodiments, a computing and/or communications network may be embodied as a wired network, wireless network, or any combinations thereof.

A computing and/or network device may include and/or may execute a variety of now known and/or to be developed operating systems, derivatives and/or versions thereof, including computer operating systems, such as Windows, iOS, Linux, a mobile operating system, such as IOS, Android, Windows Mobile, and/or the like. A computing device and/or network device may include and/or may execute a variety of possible applications, such as a client software application enabling communication with other devices. A computing and/or network device may also include executable computer instructions to process and/or communicate digital content. A computing and/or network device may also include executable computer instructions to perform a variety of possible tasks, such as browsing, searching, playing various forms of digital content, including locally stored and/or streamed video, and/or games such as, but not limited to, fantasy sports leagues. A computing and/or network device may also process input content as a prompt to one or more neural network models to provide output content. A computing and/or network device may also perform linguistic processing such as applying transforms to determine an embedding of tokens and/or apply attention models to determine service codes. The foregoing is provided merely to illustrate that claimed subject matter is intended to include a wide range of possible features and/or capabilities.

In FIG. 7, computing device 802 may provide one or more sources of executable computer instructions in the form physical states and/or signals (e.g., stored in memory states), for example. Computing device 802 may communicate with computing device 804 by way of a network connection, such as via network 808, for example. As previously mentioned, a connection, while physical, may not necessarily be tangible. Although computing device 804 of FIG. 7 shows various tangible, physical components, claimed subject matter is not limited to a computing devices having only these tangible components as other implementations and/or embodiments may include alternative arrangements that may comprise additional tangible components or fewer tangible components, for example, that function differently while achieving similar results. Rather, examples are provided merely as illustrations. It is not intended that claimed subject matter be limited in scope to illustrative examples.

Memory 822 may comprise any non-transitory storage mechanism. Memory 822 may comprise, for example, primary memory 824 and secondary memory 826, additional memory circuits, mechanisms, or combinations thereof may be used. Memory 822 may comprise, for example, random access memory, read only memory, etc., such as in the form of one or more storage devices and/or systems, such as, for example, a disk drive including an optical disc drive, a tape drive, a solid-state memory drive, etc., just to name a few examples.

Memory 822 may be utilized to store a program of executable computer instructions. For example, processor 820 may fetch executable instructions from memory and proceed to interpret and execute the fetched instructions. Memory 822 may also comprise a memory controller for accessing device readable-medium 840 that may carry and/or make accessible digital content, which may include code, and/or instructions, for example, executable by processor 820 and/or some other device, such as a controller, as one example, capable of executing computer instructions, for example. Under direction of processor 820, a non-transitory memory, such as memory cells storing physical states (e.g., memory states), comprising, for example, a program of executable computer instructions, may be executed by processor 820 and able to generate signals to be communicated via a network, for example, as previously described. Generated signals may also be stored in memory, also previously suggested. In a particular implementation, processor 820 may include general processing cores and/or specialized co-processing cores (e.g., signal processors, graphical processing unit (GPU) and/or neural network processing unit (NPU)), for example.

Memory 822 may store electronic files and/or electronic documents, such as relating to one or more users, and may also comprise a computer-readable medium that may carry and/or make accessible content, including code and/or instructions, for example, executable by processor 820 and/or some other device, such as a controller, as one example, capable of executing computer instructions, for example. As previously mentioned, the term electronic file and/or the term electronic document are used throughout this document to refer to a set of stored memory states and/or a set of physical signals associated in a manner so as to thereby form an electronic file and/or an electronic document. That is, it is not meant to implicitly reference a particular syntax, format and/or approach used, for example, with respect to a set of associated memory states and/or a set of associated physical signals. It is further noted an association of memory states, for example, may be in a logical sense and not necessarily in a tangible, physical sense. Thus, although signal and/or state components of an electronic file and/or electronic document, are to be associated logically, storage thereof, for example, may reside in one or more different places in a tangible, physical memory, in an embodiment.

Algorithmic descriptions and/or symbolic representations are examples of techniques used by those of ordinary skill in the signal processing and/or related arts to convey the substance of their work to others skilled in the art. An algorithm is, in the context of the present patent application, and generally, is considered to be a self-consistent sequence of operations and/or similar signal processing leading to a desired result. In the context of the present patent application, operations and/or processing involve physical manipulation of physical quantities. Typically, although not necessarily, such quantities may take the form of electrical and/or magnetic signals and/or states capable of being stored, transferred, combined, compared, processed and/or otherwise manipulated, for example, as electronic signals and/or states making up components of various forms of digital content, such as signal measurements, text, images, video, audio, etc.

It has proven convenient at times, principally for reasons of common usage, to refer to such physical signals and/or physical states as bits, service codes, tokens, computed likelihoods, values, elements, parameters, symbols, characters, terms, numbers, numerals, measurements, content and/or the like. It should be understood, however, that all of these and/or similar terms are to be associated with appropriate physical quantities and are merely convenient labels. Unless specifically stated otherwise, as apparent from the preceding discussion, it is appreciated that throughout this specification discussions utilizing terms such as “processing,” “computing,” “calculating,” “determining”, “establishing”, “obtaining”, “identifying”, “selecting”, “generating”, and/or the like may refer to actions and/or processes of a specific apparatus, such as a special purpose computer and/or a similar special purpose computing and/or network device. In the context of this specification, therefore, a special purpose computer and/or a similar special purpose computing and/or network device is capable of processing, manipulating and/or transforming signals and/or states, typically in the form of physical electronic and/or magnetic quantities, within memories, registers, and/or other storage devices, processing devices, and/or display devices of the special purpose computer and/or similar special purpose computing and/or network device. In the context of this particular patent application, as mentioned, the term “specific apparatus” therefore includes a general purpose computing and/or network device, such as a general purpose computer, once it is programmed to perform particular functions, such as pursuant to program software instructions.

In some circumstances, operation of a memory device, such as a change in state from a binary one to a binary zero or vice-versa, for example, may comprise a transformation, such as a physical transformation. With particular types of memory devices, such a physical transformation may comprise a physical transformation of an article to a different state or thing. For example, but without limitation, for some types of memory devices, a change in state may involve an accumulation and/or storage of charge or a release of stored charge. Likewise, in other memory devices, a change of state may comprise a physical change, such as a transformation in magnetic orientation. Likewise, a physical change may comprise a transformation in molecular structure, such as from crystalline form to amorphous form or vice-versa. In still other memory devices, a change in physical state may involve quantum mechanical phenomena, such as superposition, entanglement, and/or the like, which may involve quantum bits (qubits), for example. The foregoing is not intended to be an exhaustive list of all examples in which a change in state from a binary one to a binary zero or vice-versa in a memory device may comprise a transformation, such as a physical, but non-transitory, transformation. Rather, the foregoing is intended as illustrative examples.

Referring again to FIG. 7, processor 820 may comprise one or more circuits, such as digital circuits, to perform at least a portion of a computing procedure and/or process. By way of example, but not limitation, processor 820 may comprise one or more processors, such as controllers, microprocessors, microcontrollers, application specific integrated circuits, GPUs, NPUs, digital signal processors, programmable logic devices, field programmable gate arrays, the like, or any combination thereof. In various implementations and/or embodiments, processor 820 may perform signal processing, typically substantially in accordance with fetched executable computer instructions, such as to manipulate signals and/or states, to construct signals and/or states, etc., with signals and/or states generated in such a manner to be communicated and/or stored in memory, for example.

FIG. 7 also illustrates device 804 as including a component 832 operable with input/output devices, for example, so that signals and/or states may be appropriately communicated between devices, such as device 804 and an input device and/or device 804 and an output device. A user may make use of an input device, such as a computer mouse, stylus, track ball, microphone, scanner, keyboard, and/or any other similar device capable of receiving user actions and/or motions as input signals. Likewise, for a device having speech to text capability, a user may speak to a device to generate input signals. A user may make use of an output device, such as a display, a printer, etc., and/or any other device capable of providing signals and/or generating stimuli for a user, such as visual stimuli, audio stimuli and/or other similar stimuli.

In this context, a “neural network” as referred to herein means an architecture of a processing device defined and/or represented by a graph including nodes to represent neurons that process input signals to generate output signals, and edges connecting the nodes to represent input and/or output signal paths between and/or among neurons represented by the graph. In particular implementations, a neural network may comprise a biological neural network, made up of real biological neurons, or an artificial neural network, made up of artificial neurons, for solving artificial intelligence (AI) problems, for example. In an implementation, such an artificial neural network may be implemented by one or more computing devices such as computing devices including a central processing unit (CPU), graphics processing unit (GPU), digital signal processing (DSP) unit and/or neural processing unit (NPU), just to provide a few examples. In a particular implementation, neural network weights and/or numerical coefficients associated with edges to represent input and/or output paths may reflect gains to be applied and/or whether an associated connection between connected nodes is to be excitatory (e.g., weight with a positive value) or inhibitory connections (e.g., weight with negative value). In an example implementation, a neuron may apply a neural network weight to input signals, and sum weighted input signals to generate a linear combination.

According to an embodiment, edges in a neural network connecting nodes may model synapses capable of transmitting signals (e.g., represented by real number values) between neurons. Responsive to receipt of such a signal, a node/neural may perform some computation to generate an output signal (e.g., to be provided to another node in the neural network connected by an edge). Such an output signal may be based, at least in part, on one or more weights and/or numerical coefficients associated with the node and/or edges providing the output signal. For example, such a weight may increase or decrease a strength of an output signal. In a particular implementation, such weights and/or numerical coefficients may be adjusted and/or updated as a machine learning process progresses. In an implementation, transmission of an output signal from a node in a neural network may be inhibited if a strength of the output signal does not exceed a threshold value.

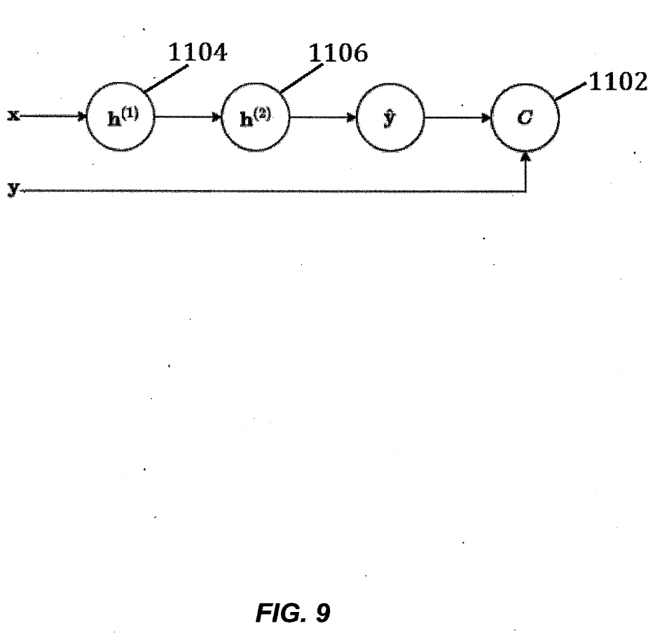

FIG. 9 is a schematic diagram of a neural network 1000 formed in “layers” in which an initial layer is formed by nodes 1002 and a final layer is formed by nodes 1006. All or a portion of features of NN 1000 may be implemented in aspects of neural network model 504, cloud prediction model 112 or cloud computing system 240, for example. Neural network (NN) 1000 may include an intermediate layer formed by nodes 1004. Edges shown between nodes 1002 and 1004 illustrate signal flow from an initial layer to an intermediate layer. Likewise, edges shown between nodes 1004 and 1006 illustrate signal flow from an intermediate layer to a final layer. While neural network 1000 shows a single intermediate layer formed by nodes 1004, it should be understood that other implementations of a neural network may include multiple intermediate layers formed between an initial layer and a final layer.

According to an embodiment, a node 1002, 1004 and/or 1006 may process input signals (e.g., received on one or more incoming edges) to provide output signals (e.g., on one or more outgoing edges) according to an activation function. An “activation function” as referred to herein means a set of one or more operations associated with a node of a neural network to map one or more input signals to one or more output signals. In a particular implementation, such an activation function may be defined based, at least in part, on a weight associated with a node of a neural network. Operations of an activation function to map one or more input signals to one or more output signals may comprise, for example, identity, binary step, logistic (e.g., sigmoid and/or soft step), hyperbolic tangent, rectified linear unit, Gaussian error linear unit, Softplus, exponential linear unit, scaled exponential linear unit, leaky rectified linear unit, parametric rectified linear unit, sigmoid linear unit, Swish, Mish, Gaussian and/or growing cosine unit operations. It should be understood, however, that these are merely examples of operations that may be applied to map input signals of a node to output signals in an activation function, and claimed subject matter is not limited in this respect. Additionally, an “activation input value” as referred to herein means a value provided as an input parameter and/or signal to an activation function defined and/or represented by a node in a neural network. Likewise, an “activation output value” as referred to herein means an output value and/or signal provided by an activation function defined and/or represented by a node of a neural network. In a particular implementation, an activation output value may be computed and/or generated according to an activation function based on and/or responsive to one or more activation input values received at a node. In a particular implementation, an activation input value and/or activation output value may be structured, dimensioned and/or formatted as “tensors”. Thus, in this context, an “activation input tensor” or “input tensor” as referred to herein means an expression of one or more activation input values according to a particular structure, dimension and/or format. Likewise in this context, an “activation output tensor” or “output tensor” as referred to herein means an expression of one or more activation output values according to a particular structure, dimension and/or format.

According to an embodiment, neural network 1000 may be characterized as having a particular structure or topology based on, for example, a number of layers, number of nodes in each layer, activation functions implemented at each node, quantization of weights and quantization of input/output activations. Neural network 1000 may be further characterized by weights to be assigned to nodes to affect activation functions at respective nodes. During execution, neural network 1000 may be characterized as having a particular state or “intermediate state” determined based on values/signals computed by nodes (e.g., as activation values to be provided to nodes in a subsequent layer of nodes and/or an output tensor).

In particular implementations, neural networks may enable improved results in a wide range of tasks, including image recognition, speech recognition, content generation, just to provide a couple of example applications. To enable performing such tasks, features of a neural network (e.g., nodes, edges, weights, layers of nodes and edges) may be structured and/or configured to form “filters” that may have a measurable/numerical state such as a value of an output signal. Such a filter may comprise nodes and/or edges arranged in “paths” and are to be responsive to sensor observations provided as input signals. In an implementation, a state and/or output signal of such a filter may indicate and/or infer detection of a presence or absence of a feature in an input signal.

In particular implementations, intelligent computing devices to perform functions supported by neural networks may comprise a wide variety of stationary and/or mobile devices, such as, for example, automobile sensors, biochip transponders, heart monitoring implants, Internet of things (IoT) devices, kitchen appliances, locks or like fastening devices, solar panel arrays, home gateways, smart gauges, robots, financial trading platforms, smart telephones, cellular telephones, security cameras, wearable devices, thermostats, Global Positioning System (GPS) transceivers, personal digital assistants (PDAs), virtual assistants, laptop computers, personal entertainment systems, tablet personal computers (PCs), PCs, personal audio or video devices, personal navigation devices, just to provide a few examples.

According to an embodiment, a neural network may be structured in layers such that a node in a particular neural network layer may receive output signals from one or more nodes in an upstream layer in the neural network, and provide an output signal to one or more nodes in a downstream layer in the neural network. One specific class of layered neural networks may comprise a convolutional neural network (CNN) or space invariant artificial neural networks (SIANN) that enable deep learning. Such CNNs and/or SIANNs may be based, at least in part, on a shared-weight architecture of a convolution kernels that shift over input features and provide translation equivariant responses. Such CNNs and/or SIANNs may be applied to image and/or video recognition, recommender systems, image classification, image segmentation, medical image analysis, natural language processing (e.g., medical records processing), brain-computer interfaces, financial time series, just to provide a few examples.

Another class of layered neural network may comprise a recursive neural network (RNN) that is a class of neural networks in which connections between nodes form a directed cyclic graph along a temporal sequence. Such a temporal sequence may enable modeling of temporal dynamic behavior. In an implementation, an RNN may employ an internal state (e.g., memory) to process variable length sequences of inputs. This may be applied, for example, to tasks such as unsegmented, connected handwriting recognition or speech recognition, just to provide a few examples. In particular implementations, an RNN may emulate temporal behavior using finite impulse response (FIR) or infinite impulse response (IIR) structures. An RNN may include additional structures to control stored states of such FIR and IIR structures to be aged. Structures to control such stored states may include a network or graph that incorporates time delays and/or has feedback loops, such as in long short-term memory networks (LSTMs) and gated recurrent units.

According to an embodiment, output signals of one or more neural networks (e.g., taken individually or in combination) may at least in part, define a “predictor” to generate prediction values associated with some observable and/or measurable phenomenon and/or state. In an implementation, a neural network may be “trained” to provide a predictor that is capable of generating such prediction values based on input values (e.g., measurements and/or observations) optimized according to a loss function. For example, a training process may employ backpropagation techniques. “Backpropagation,” as referred to herein, is to mean a process of fitting parameters of a trained inference model such a model comprising one or more neural networks. In fitting parameters of a neural network, for example, backpropagation is to compute a gradient of a loss function with respect to the weights of the neural network. Based on such a computed gradient of a loss function, weights may be updated so as to minimize and/or reduce such a loss function. In one particular implementation, a gradient descent of a loss function, or variants such as stochastic gradient descent of a loss function, may be used. In training parameters of a neural network, backpropagation may comprise computing a gradient of a loss function with respect to individual weights by the chain rule, computing a gradient one layer at a time, iterating backward from the last layer to avoid redundant calculations of intermediate terms in the chain rule, for example. It should be understood, however, that this is merely an example of how a process of backpropagation may be applied, and claimed subject matter is not limited in this respect. In particular implementations, backpropagation may be used to iteratively update neural network weights to be associated with nodes and/or edges of a neural network based, at least in part on “training sets.” Such training sets may include training measurements and/or observations to be supplied as input values that are paired with “ground truth” observations. Based on a comparison of such ground truth observations and associated prediction values generated based on such input values in a training process, weights may be updated according to a loss function using backpropagation. FIG. 9 is a flow diagram of an aspect of a training operation employing backpropagation to train parameters for a feedforward neural network, according to an embodiment. It should be understood, however, that this is merely an example of a type of neural network that may be trained using backpropagation, and that similar backpropagation techniques may be applied to train parameters of other types of neural networks without deviating from claimed subject matter. Training sets may be provided to such a training operation as pairs of vectors (x,y) where x is an input vector and y is a corresponding ground truth label. Input vector x may be provided as an input tensor to a first hidden layer 1104 to produce an output vector h⁽¹⁾, which is provided as an input to a second hidden layer 1106 to provide an output vector h⁽²⁾. An inference and/or prediction ŷ may be computed based, at least in part, on the output vector h⁽²⁾. A loss value C may be computed at 1102 according to one or more loss functions based, at least in part, on inference and/or prediction y and ground truth label y.

In the particular embodiment of FIG. 9, inference and/or prediction ŷ, and output vectors h⁽¹⁾ and h⁽²⁾ may be modelled as follows:

$${h}^{( 1 )} = {g}^{( 1 )} ( {W}^{( 1 ) T} x + {b}^{( 1 )} )$$

$${h}^{( 2 )} = {g}^{( 2 )} ( {W}^{( 2 ) T} {h}^{( 1 )} + {b}^{( 2 )} )$$

$$\hat{y} ( x ) = {W}^{( 3 ) T} {h}^{( 2 )} + {b}^{( 3 )} ,$$

where:
g⁽ⁱ⁾ is an activation function applied at nodes in hidden layer i;
W⁽ⁱ⁾ is a matrix of weights such that weight

$${W}_{j k}^{( i )}$$

is to be applied at an edge going from node j in layer i−1 to node k in hidden layer i; and
b⁽ⁱ⁾ is a bias matrix applied at hidden layer i.

In a particular implementation in which a feedforward neural network includes three or more hidden layers, computation of ŷ(x) may be generalized as follows:

$$\hat{y} ( x ) = {W}^{( N ) T} {h}^{( N - 1 )} + {b}^{( N )} .$$

Loss value C(y, ŷ) may be computed according to any one of several formulations of a loss function include, for example, a means square error loss or mean absolute error loss, just provide a couple of examples of a loss function. In a particular implementation, a loss function to compute C(y, ŷ) may be differentiable such that

$$\frac{\partial C}{\partial {W}_{jk}^{( i )}}$$

may be determined using the chain rule and may be computed for any weight

$${W}_{j k}^{( i )} .$$

According to an embodiment, values for W⁽ⁱ⁾ may be determined iteratively for training sets (x,y) using a gradient descent technique.

In this context, a “supervised operation” or “unsupervised operation” as referred to herein are to mean a machine-learning operation in which training sets provided as inputs for training iterations are paired with “ground truth” labels. In a training iteration/epoch of such a supervised operation, for example, a loss value may be computed based, at least in part, on an inference computed by a trainable model based on one or more input values a training set and a ground truth label in the training set paired with the one or more input values. For example, a supervised operation may execute a loss function to compute a loss value based, at least in part, on a comparison of a computed inference and ground truth observations/values paired with the computed inference. In this context, a “self-supervised operation” as referred to herein is to mean a machine-learning operation in which input training sets are provided without “ground truth” labels. In a training iteration/epoch of such a self-supervised operation, for example, a loss function may compute a loss value based, at least in part, on an inference computed based on a training set and in the absence of any ground truth label paired with the training set.

One particular embodiment disclosed herein is directed to a method, comprising: training parameters of one or more computing devices to predict a particular irrigation event to result in a particular target soil moisture level for a particular crop field and/or client farmer, wherein: the particular irrigation event is to be predicted based, at least in part, on time-referenced observations and/or measurements comprising observations and/or measurements of: water pressure and/or flow of water to irrigate the particular crop field; one or more electrical power signals applied to at least a portion of the flow of water to reduce a surface tension in the flow of water; and soil moisture in the crop field, wherein training the parameters of the one or more computing devices further comprises: computing a loss function based, at least in part, on computed prediction of one or more soil moisture levels in the particular crop field and time-referenced observations and/or measurements of soil moisture and/or soil nutrients in the particular crop field; and updating the parameters of the one or more computing devices based, at least in part, on a gradient of the loss function computed over multiple training epochs.

In the preceding description, various aspects of claimed subject matter have been described. For purposes of explanation, specifics, such as amounts, systems and/or configurations, as examples, were set forth. In other instances, well-known features were omitted and/or simplified so as not to obscure claimed subject matter. While certain features have been illustrated and/or described herein, many modifications, substitutions, changes and/or equivalents will now occur to those skilled in the art. It is, therefore, to be understood that the appended claims are intended to cover all modifications and/or changes as fall within claimed subject matter.

What is claimed is:

1. A method comprising:
collecting time-referenced observations and/or measurements from sensors deployed in a crop field, the crop field having been irrigated by a flow of water processed by application of one or more electrical power signals to at least a portion of the flow of water to reduce a surface tension in the flow of water; and
computing a recommended irrigation schedule to achieve a target soil moisture based, at least in part, on the time-referenced observations and/or measurements, wherein the time-referenced observations and/or measurements further comprise observations and/or measurements of: water pressure and/or flow of water; two or more attributes of the one or more electrical power signals and soil moisture in the crop field.

2. The method of claim 1, wherein the measurements and/or observations of the two or more attributes of one or more electrical power signals comprise measurements and/or observations of a signal current or a signal frequency, or a combination thereof.

3. The method of claim 1, wherein the recommended irrigation schedule is further based, at least in part, on a weather history, weather forecast or almanac, or a combination thereof.

4. The method of claim 1, wherein the time-referenced observations and/or measurements from the sensors deployed in the crop field comprise measurements and/or observations of ground moisture, air temperature, ground temperature, air humidity, sunlight intensity or wind, or a combination thereof.

5. The method of claim 1, wherein computing the recommended irrigation schedule further comprises executing a trained neural network model to compute the recommended irrigation schedule based, at least in part, on the target soil moisture.

6. The method of claim 5, wherein the recommended irrigation schedule is computed based, at least in part, on execution of a neural network model trained by training sets for a particular farmer client comprising:
time-referenced observations and/or measurements collected from sensors deployed in the crop field over multiple growing seasons, the observations and/or measurements collected from sensors deployed in the crop field over multiple growing seasons comprising at least observations and/or measurements of soil moisture and/or soil nutrients in the crop field;
time-referenced observations and/or measurements of a flow of water for irrigation of the crop field; and
time-referenced observations and/or measurements of two or more attributes of one or more electrical power signals applied to at least a portion of the flow of water to reduce a surface tension in the flow of water.

7. The method of claim 6, wherein the observations and/or measurements of soil moisture are obtained from multiple soil depths.

8. The method of claim 5, wherein parameters of the trained neural network model are updated in training epochs comprising computation of a loss function based, at least in part, on predictions of soil moisture and time-referenced observations and/or measurements of soil moisture and/or soil nutrients.

9. The method of claim 1, wherein the recommended irrigation schedule comprises one or more irrigation events.

10. The method of claim 9, wherein at least one of the irrigation events is defined, at least in part, by an irrigation start time, irrigation duration or an irrigation flow, or a combination thereof.

11. A method, comprising:
training parameters of one or more computing devices to predict a particular irrigation event to result in a particular target soil moisture level for a particular crop field and/or client farmer, wherein: the particular irrigation event is to be predicted based, at least in part, on time-referenced observations and/or measurements comprising observations and/or measurements of: water pressure and/or flow of water to irrigate the particular crop field; two or more attributes of one or more electrical power signals applied to at least a portion of the flow of water to reduce a surface tension in the flow of water; and soil moisture in the crop field, wherein training the parameters of the one or more computing devices further comprises:
computing a loss function based, at least in part, on one or more time-referenced target soil moisture levels for the particular crop field and one or more time-referenced observations and/or measurements of soil moisture and/or soil nutrients in the particular crop field; and
updating the parameters of the one or more computing devices based, at least in part, on a gradient of the loss function computed over multiple training epochs.

12. The method of claim 11, wherein the measurements and/or observations of the two or more attributes of the one or more electrical power signals comprise measurements and/or observations of a signal current or a signal frequency, or a combination thereof.

13. The method of claim 11, wherein the observations and/or measurements of soil moisture are obtained from multiple soil depths.

14. The method of claim 11, wherein the loss function is further computed based, at least in part, on computed prediction of one or more soil moisture levels in the particular crop field.

15. An apparatus comprising:
one or more communication devices; and
one or more processors coupled to the one or more communication devices to:
obtain, from one or more messages received at the one or more communication devices, time-referenced observations and/or measurements collected from sensors deployed in a crop field, the crop field having been irrigated by a flow of water processed by application of one or more electrical power signals to at least a portion of the flow of water to reduce a surface tension in the flow of water; and
compute a recommended irrigation schedule to achieve a target soil moisture based, at least in part, on the time-referenced observations and/or measurements, wherein the time-referenced observations and/or measurements further comprise observations and/or measurements of: water pressure and/or flow of water; the one or more electrical power signals and soil moisture in the crop field.

16. The apparatus of claim 15, wherein the measurements and/or observations of the one or more electrical power signals comprise measurements and/or observations of a signal current or a signal frequency, or a combination thereof.

17. The apparatus of claim 15, wherein the recommended irrigation schedule is further based, at least in part, on a weather history, weather forecast or almanac, or a combination thereof.

18. The apparatus of claim 15, wherein the time-referenced observations and/or measurements from the sensors deployed in the crop field comprise measurements and/or observations of ground moisture, air temperature, ground temperature, air humidity, sunlight intensity or wind, or a combination thereof.

19. The apparatus of claim 15, wherein computing the recommended irrigation schedule further comprises executing a trained neural network model to compute the recommended irrigation schedule based, at least in part, on the target soil moisture.

20. The apparatus of claim 19, wherein the trained neural network model is trained by training sets for a particular farmer client comprising:
time-referenced observations and/or measurements collected from sensors deployed in the crop field over multiple growing seasons, the observations and/or measurements collected from sensors deployed in the crop field over multiple growing seasons comprising at least observations and/or measurements of soil moisture and/or soil nutrients in the crop field;
time-referenced observations and/or measurements of a flow of water for irrigation of the crop field; and
time-referenced observations and/or measurements of two or more attributes of one or more electrical power signals applied to at least a portion of the flow of water to reduce a surface tension in the flow of water.
